(19) United States
(12) Patent Application Publication
(10) Pub. No.: US 2009/0175928 A1
(43) Pub. Date: Jul. 9, 2009

(54) Lip Plaster

(76) Inventors: Stephan Maier, Leverkusen (DE); Ulrike Vollmer, Dormagen (DE)

Correspondence Address: FRIEDRICH KUEFFNER, 317 MADISON AVENUE, SUITE 910, NEW YORK, NY, 10017, US

(21) Appl. No.: 12/083,736

(22) PCT Filed: Oct. 9, 2006
(86) PCT No.: PCT/DE2006/001768

§ 371 Date: Apr. 18, 2008

(30) Foreign Application Priority Data
Oct. 20, 2005 (DE) 10 2005 050 654.2

(51) Int. Cl. A61K 9/70; A61P 31/12; A61K 31/52; A61K 31/045; A61K 31/727; A61K 33/00; A61K 33/30; A61K 36/28

(52) U.S. Cl. 424/449; 514/263.38; 514/724; 514/56; 424/600; 424/642; 424/737

(57) ABSTRACT

The invention relates to a self-adhesive lip plaster which comprises at least one single-layered matrix and which contains at least one antiviral active medical substance for using in the event of Herpes labialis. Said matrix is arranged on a flexible carrier film which is hydrophobic and also permeable to vapor.

The invention concerns a self-adhesive topical application system that contains an active ingredient for use as a lip patch in acute infections with herpes simplex viruses, especially in labial or perioral infections with human herpesvirus 1.

More than 90% of the population carry human herpesvirus 1, and herpes labialis appears as a clinical manifestation in about 20-40% of the virus carriers.

The initial infection with herpesviruses of type 1 usually occurs in childhood and is usually asymptomatic. The virus is transmitted by droplet infection or smear infection on injured skin or mucous membrane.

After the initial contact, the virus migrates into the epithelial cells along sensory nerves to the ganglia. The virus remains latent in the ganglia until certain factors, such as stress, UV light, fever, or nausea, set the replication mechanism of the virus in motion again. New viruses are formed and migrate along the nerve paths and into the skin cells. The typical symptoms then appear in the skin: tingling sensation, itchiness, and a feeling of tension are followed after one to two days by vesicular lesions on a reddened background. The vesicles dry in the following days to form crusts, which slowly heal. The affected area is normally smaller than 100 mm² with 3 to 5 blisters.

A herpes outbreak often has a strong negative effect on the quality of life of the affected individual. The frequency of these outbreaks is extremely variable, ranging from rare episodes every 5 to 10 years to monthly episodes or even more frequent outbreaks.

Previous drug treatment of herpes labialis has involved either topical ointments and creams or oral tablets and capsules. Examples of active substances used for topical treatment are docosanol, tromcardin HCR, zinc sulfate, combinations of zinc sulfate and heparin Na, silicic acid, melissa extract, aciclovir, and penciclovir. The following is an example of a dosage recommendation for the use of topical agents that contain penciclovir: for herpes of the lips, it should be applied every 2 hours (at least 6-8 times daily, and, if at all possible, 10-12 times on the second day of treatment). This high frequency of application makes it difficult to realize patient compliance. Compliance with this application recommendation means considerable interference with one's normal daily routine. In this connection, especially the nighttime treatment is a problem, because a two-hour application interval cannot be maintained without considerable reduction of one's quality of life. In addition, ointments or creams applied at night are often quickly wiped off on one's nightclothes or bedclothes. These dose-free intervals can contribute to a significant prolongation of the episode.

Examples of active substances given as oral treatment include aciclovir, valaciclovir, famciclovir, and foscarnet. For the oral treatment of an acute outbreak, we find, for example, the following dosage recommendation for aciclovir: 200-400 mg 5 times daily for 5 days. Here again, a high frequency of administration is required, and this has a strong influence on the daily planning of the patient. Not only must the patient think about taking his medication at regular intervals, but also he must keep the medication with him and have a drink available for taking the medication.

Persons who are suffering from an acute herpes attack should, if at all possible, avoid direct contact with other persons. Even the shared use of glasses or dinnerware can lead to transmission of the virus. To prevent the virus from being transferred to other parts of the body or to other persons, the focus of infection should not be touched with the fingers. For the same reason, topical preparations should be applied with a cotton swab. However, patients often fail to do this, mainly because they would have to carry a large number of these “application aids” with them everywhere they go due to the short dosage intervals.

A self-adhesive application system that contains active ingredients is already known from DE 10 2005 003 387 A1. This application system has a backing layer that contains a matrix that consists of a hydrocolloid. A hydrocolloid used for this purpose comprises, for example, polysaccharides and proteins, which can dissolve in water as colloids and have a strong capacity for gel formation. Therefore, hydrocolloids of this type are unsuitable for use in lip patches for the simple reason that hydrocolloids do not have hydrophobic properties. In addition, when hydrocolloids are used, contour stability is not ensured. This shortcoming has negative effects for a cosmetic and physical covering. Moreover, a swollen hydrocolloid offers an optimum nutrient medium for microorganisms, so that there is a risk of inflammation in the area of application.

DE 198 56 101 A1 describes a patch preparation for local administration of active substances in the oral cavity. Accordingly, this is a mouth patch with a drug-containing matrix, which is applied in the oral cavity and releases the drug in the oral cavity. The mouth patch with a drug-containing matrix has an adhesive layer for attaching the patch to the oral mucosa and a drug-containing layer. Both layers are designed to be at least partially water-soluble.

US 2004/0126333 A1 describes another patch for use in the oral cavity. The patch is designed as a single-layer film.

EP 0 381 193 discloses another patch for application to the oral mucosa.

WO 00/04884 describes a drug preparation for the topical treatment of mucocutaneous herpes infections and herpetic keratitis.

WO 2005/016321 discloses a mucoadhesive therapeutic system with a backing layer made of a water-soluble polymer.

WO 95/00184 describes a foam that is solid and yet flexible. The foam provides a porous system that serves to absorb a liquid medium and in the process forms a hydrogel.

The objective of the present invention is to provide a self-adhesive topical patch that can be used in the treatment of herpes labialis and avoids the disadvantages of previously used oral and topical forms of administration.

In this connection, the lip presents a special challenge as a site of application. On the one hand, the patch must have sufficient adhesive strength to guarantee good adhesion despite constant mechanical stress (talking) and contact with hot and cold liquids, fats, etc. (eating). On the other hand, at the end of the application interval, it must be possible to remove the patch without causing pain and without injuring the infected area of skin beneath the patch. In addition, a feeling that a foreign body is present is to be avoided as far as possible.

In accordance with the invention, this objective is achieved with a topical, self-adhesive lip patch with a matrix that comprises at least one layer. The lip patch of the invention is characterized by the fact that the matrix contains at least one antiviral drug for the treatment of herpes labialis and that the matrix is arranged on a flexible backing film that is both water-repellent and permeable to water vapor.

The choice of suitable adjuvants and suitable types of films is especially important, as the following example of a wearing test on 5 test subjects demonstrates.

TABLE 1
EFFECT OF THE COVER LAYER ON THE WEARING
TIME AND THE SENSATION OF A FOREIGN BODY,
TESTED ON 5 TEST SUBJECTS.

| | Average Wearing Time | Foreign Body Sensation |
|---|---|---|
| | [h] | [1-6]* |
| Polyurethane 50 | 12.4 | 2.25 |
| μm | | |
| Polyethylene 75 | 8.0 | 3.5 |
| μm | | |

*1 = very good, 2 = good, 3 = satisfactory, 4 = adequate, 5 = deficient, 6 = unsatisfactory

The use of a self-adhesive topical patch makes it possible to achieve a high local concentration of active substance over a long period of time. For aciclovir, for example, it is known that the active substance must be made available quickly and in a sufficient amount if therapeutic success is to be achieved. In addition, it is simpler and more pleasant for the patient if he needs to think about the application of a patch only 1-3 times per 24 hours instead of having to apply an ointment or cream 6-12 times per day or having to take a tablet 5 times per day. The average application time of the self-adhesive patch of 8 hours significantly increases patient compliance and thus contributes greatly to therapeutic success.

To test the acceptance and efficacy of a herpes patch, an independent observation of use was carried out on 6 volunteers with herpes infection with a first prototype (with 4 mg of aciclovir per patch). 50% of the participants rated the efficacy of the patch as clearly better than the drug they otherwise used, and one of the participants rated the efficacy of the patch as worse.

TABLE 2
RESULTS OF THE OBSERVATION
OF USE IN 6 VOLUNTEERS.

| | Patches | Average Time | Foreign Body | Efficacy |
|---|---|---|---|---|
| | Worn | Worn [h] | Sensation [1-6]* | [1-6]* |
| Person 1 | 5 of 5 | 7 | 2 | 2 |
| Person 2 | 5 of 5 | 8 | 3 | 3 |
| Person 3 | 5 of 5 | 9 | 2 | 3 |
| Person 4 | 5 of 5 | 15 | 2 | 2 |
| Person 5 | 5 of 5 | 8 | 3 | 2 |
| Person 6 | 5 of 5 | 14 | 2 | 2 |

*1 = very good, 2 = good, 3 = satisfactory, 4 = adequate, 5 = deficient, 6 = unsatisfactory

The cosmetic effect of the patch can also be seen as an advantage. The thin, flexible patch can contain one or more coloring pigments. This makes it possible to conceal the lesions, which are usually rather unattractive. Ideally, makeup can also be applied over the patch. Another decisive advantage of the covering of the infected area of skin by the patch is that this makes it possible to avoid spreading or transmitting the viral infection by droplet infection or smear infection. A tingling sensation, itchiness, and a feeling of tension often cause patients to touch the infected areas of skin with their fingers and in this way spread the infection. The patch prevents direct contact with the focus of infection. In addition, this facilitates healing of the wounds after the vesicular phase.

All possible drugs can be used, for example, docosanol, tromcardin HCl, zinc sulfate, heparin Na, silicic acid, melissa extract, aciclovir and penciclovir, alone or in combination. The patch can also contain other substances for other types of indications, such as wound healing promoters, e.g., dexpanthenol.

The self-adhesive topical patch of the invention is a single-layer or multilayer matrix system, which either consists of only one matrix with at least two layers or which has, besides a single-layer or multilayer matrix, a moisture-resistant and impermeable cover layer, and a protective layer that can be pulled off. Possible components of the impermeable cover layer are polyester, polypropylene, polyurethane, ethylene-vinyl acetate, or polyethylene. The removable protective layer may consist of polyester, polypropylene, polysiloxane, polyacrylate, ethylene-vinyl acetate, polyurethane, polyisobutene, or paper coated with silicone and/or polyethylene.

A special embodiment of the invention comprises a two-layer matrix system that contains neither a cover layer nor a protective layer. This two-layer system can consist of a water-insoluble polymer layer, in which a wide variety of adjuvants can be incorporated, and of a second polymer layer that is not self-adhesive, is activated by moisture, is water-soluble, and contains one or more active ingredients and other adjuvants.

Standard matrix formers for medical applications can be used alone or in combination, such as polyacrylate, silicone, polyisobutylene, rubber, rubber-like synthetic homopolymers, copolymers, or block polymers, butyl rubber, styrene-isoprene copolymer, polyurethanes, copolymers of ethylene, polysiloxanes, or styrene-butadiene copolymer.

The matrix formers based on silicone can be silicone adhesives that are based on two main components: a polymer or adhesive, especially polysiloxane, and a tackifying resin. The polysiloxane adhesive is usually formulated with a crosslinking agent for the adhesive, typically a high-molecular-weight polydiorganosiloxane, and with the resin to provide a three-dimensional silicate structure via an appropriate organic solvent. Admixing the resin with the polymer is the most important factor for modifying the physical properties of the polysiloxane adhesive; cf. for example, Sobieski et al., “Silicone Pressure Sensitive Adhesives”, Handbook of Pressure Sensitive Adhesive Technology, 2nd edition, pp. 508-517 (D. Satas, Editor), Van Nostrand Reinhold, New York (1989).

Another example of a pressure-sensitive adhesive based on silicone is trimethylated silicon dioxide treated with polydimethylsiloxane with terminal trimethylsiloxy groups.

Moreover, the use of silicone elastomers has been found to be especially suitable. These crosslinked elastomers, which are often called soft skin adhesives, are addition products of polydimethylsiloxane with a terminal vinyl group and siloxanes with hydrogen groups. These adhesives have the special property that they can be removed easily and painlessly from the skin and/or lip. In addition, the high degree of flexibility, the good permeability to moisture, and the reduced adhesion on skin that is not intact are found to be very advantageous for the solution to the problem on which the invention is based.

The matrix formers based on acrylate can be any desired homopolymer, copolymer, or terpolymer consisting of different acrylic acid derivatives.

For example, the acrylate polymers can be polymers of one or more monomers of acrylic acids and other copolymerizable monomers. In addition, the acrylate polymers can comprise copolymers of alkyl acrylates and/or alkyl methacrylates and/or copolymerizable secondary monomers or monomers with functional groups. If the amount of each type that is added as a monomer is changed, the cohesive properties of the resulting acrylate polymers can be changed. In general, the acrylate polymer consists of at least 50 wt. % of an acrylate, methacrylate, alkyl acrylate, or alkyl methacrylate monomer, 0 to 20 wt. % of a functional monomer that can be copolymerized with acrylate, and 0 to 40 wt. % of another monomer.

Acrylate monomers are listed below which can be used with acrylic acid, methacrylic acid, butyl methacrylate, hexyl acrylate, hexyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate and tridecyl methacrylate.

For example, functional monomers that are copolymerizable with the aforementioned acrylates, methacrylates, alkyl acrylates or alkyl methacrylates can be used, e.g., acrylic acid and methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, and methoxyethyl methacrylate.

Further details and examples of pressure-sensitive adhesive acrylates that are suitable for the invention are described in Satas' Handbook of Pressure Sensitive Adhesive Technology “Acrylic Adhesives”, 2nd edition, pp. 396-456 (D. Satas, Editor), Van Nostrand Reinhold, New York (1989).

The following components and/or all mixtures thereof can be used as permeation promoters: monovalent and/or polyvalent aliphatic, alicyclic, and/or aromatic-aliphatic alcohols with up to eight C atoms each, e.g., ethanol, 1,2-propanediol, dexpanthenol, and/or polyethylene glycol; alcohol/water mixtures; saturated and/or unsaturated fatty alcohols with 8-18 C atoms each; terpenes, e.g., cineol, carveol, menthone, terpineol, verbenone, menthol, limonene, thymol, cymene, terpinene-4-ol, neomenthol, geraniol, fenchone; mixtures of terpenes and ethanol and/or propylene glycol; tea tree oil; saturated and/or unsaturated cyclic ketones; alkyl methyl sulfoxides; saturated and/or unsaturated fatty acids with 8-18 C atoms each; their esters and salts; natural vitamin E; synthetic vitamin E and/or vitamin E derivatives; sorbitan fatty acid esters and ethoxylated sorbitan fatty acid esters; azones (laurocapram); azones mixed with alcohols; urea; 1-alkylpyrrolidone; block copolymers of polyethylene glycol and dimethylsiloxane with a cationic group at one end; folate polyethylene glycol liposome, proliposome; polyoxyethylene-10-stearyl ether; a mixture of polyoxyethylene-10-stearyl ether and glyceryl dilaurate; dodecyl-2-(N,N-dimethylamino)-propanol tetradecanoate and/or dodecyl-2-(N,N-dimethylamino)-propionate; N-acetylprolinate esters with more than 8 C atoms; nonionic surfactants, e.g., lauryl ether, esters of polyoxyethylene; ethosome (phospholipids vesicle); dimethyl(arylimino)sulfurane; a mixture of oleic acid analogs and propylene glycol; and a mixture of Padimate O, octyl salicylate, octyl methoxycinnamate, and laurocapram.

The invention is explained in greater detail by the examples below but is not limited to these examples.

### EXAMPLE 1

Two-layer matrix patch without a cover layer or protective layer, adhesive side moisture activated.

First Layer (adhesive side):

6 g of Carbopol 971 are homogenized in 90 g of 96% ethanol. 3.6 g of Lutrol F 127, 60 g of purified water, 3.6 g of propylene glycol, and 5.4 g of aciclovir are added to the homogeneous polymer solution. The homogeneous compound is spread on a nonsiliconized PET film (e.g., 100 μm) and dried.

Second Layer:

4.8 g of ethyl cellulose are dissolved in 40 g of 96% ethanol. 2.4 g of castor oil, 0.12 g of polysorbate 80, 1.8 g of gum arabic, and coloring pigments are added. The colored coating compound is spread on the dried first layer and dried. Patches with a surface area of 1 cm² are punched from the two-layer laminate.

### EXAMPLE 2

One-layer matrix patch with a cover layer but no protective layer, adhesive side moisture activated

6 g of Carbopol 971 are homogenized in 54 g of 96% ethanol. 5.4 g of aciclovir, 12 g of purified water, 0.6 g of glycerol, and coloring pigments are added to the homogeneous polymer solution. The homogeneous compound is spread on a polyurethane film (e.g., 50 μm) or on a polyethylene film and dried. Patches with a surface area of 1 cm² are punched out.

### EXAMPLE 3

One-layer matrix patch with a cover layer and a protective layer

4.8 g each of the two components of a silicone elastomer (e.g., 7-9800 Soft Skin Part A and Part B, Dow Corning Corp.) are mixed. 0.29 g of Aerosil and 4.8 g of an amine-compatible silicone adhesive (e.g., BIOPSA 7-4302, Dow Corning Corp.) are added to the mixture, and then 2.4 g of aciclovir and coloring pigments are added. The homogeneous coating solution is spread on a polyurethane film (e.g., 50 μm) or on a polyethylene film and dried. The removable protective layer (e.g., a polyester film coated with a fluoropolymer) is laminated on the matrix side. Patches with a surface area of 1 cm² are punched out.

The essential properties of the lip patch of the invention will now be summarized. The lip patch has a water-repellent backing layer to enable it to be worn on the lip for several hours. The lip patch maintains its thin and flexible characteristics and its contour stability the entire time it is worn. The contour stability guarantees that during the entire period of use, the herpes lesion is physically and cosmetically covered.

The thin, flexible backing film used for the lip patch is soft and has water-repellent and semiocclusive properties. Polyurethane films are especially suitable. A backing film made of polyurethane is semipermeable and provides reliable protection against microorganisms and wetness. Due to semipermeable nature of the backing film, however, gas exchange is possible, so that especially excessive water vapor can escape. The herpes lesion can thus quickly and gently heal in a moist but not wet environment.

As a result of the structural realization of the lip patch, it is guaranteed that when it is applied to the lip or to areas of skin surrounding the lip, it releases the drug locally to the lip or to the areas of skin surrounding the lip.

The lip patch of the invention makes possible especially a concentration of active substance of greater than 40 wt. %. In particular, it is proposed that the lip patch be constructed with at least two layers, with at least one of the layers being realized as a polymer film. The polymer film provides an adhesive layer that can be realized thin and bubble-free. This helps achieve good adhesion of the system at the site of application. Especially adhesion over the full area of application of the patch on the affected skin area helps achieve the desired diffusion of the drug into the intended site of action.

To help achieve the desired cosmetic effect, it is especially contemplated that the cover layer provided by the backing film be realized as an opaque or stained structure. Pigmented or painted embodiments are also possible.

The aforementioned drug load of at least 40 wt. % makes it possible to achieve a high concentration of active substance at the site of action for the entire time the patch is worn and thus for a period of at least eight hours. Despite this high drug load, the self-adhesive matrix has extremely good adhesive strength.

Specific embodiments of the invention are shown schematically in the drawings.

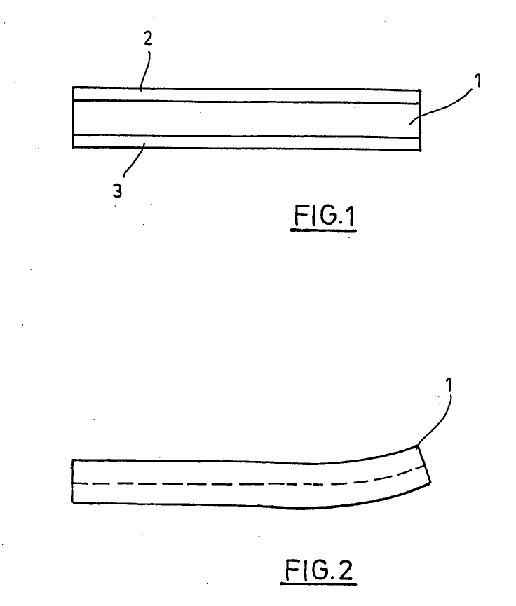

FIG. 1 shows a topical patch with a cover layer and a protective layer.

FIG. 2 shows a topical patch with a two-layer matrix without a protective layer or cover layer.

FIG. 1 shows an embodiment of a topical patch that has already been described. The patch has a matrix 1 with at least one layer. One of the surfaces of the matrix 1 is furnished with a cover layer 2, and the surface on the other side is furnished with a protective layer 3. After the protective layer 3 has been pulled off, the generally self-adhesive matrix 1 can be adhesively attached to the application site. The cover layer 2 makes it possible for makeup to be applied over the adhesively attached topical patch.

FIG. 2 shows the embodiment of a two-layer matrix 1 without a cover layer 2 or a protective layer 3. One of the matrix layers is water-insoluble, and the other is water-soluble and activated by moisture.

1. A topical, self-adhesive lip patch with a matrix that comprises at least one layer, wherein the matrix contains at least one antiviral drug for the treatment of herpes labialis and that the matrix is arranged on a flexible backing film that is both water-repellent and permeable to water vapor.

2. A topical patch in accordance with claim 1, wherein a combination of at least two antiviral drugs is used.

3. A topical patch in accordance with claim 1, wherein the patch is designed to provide cosmetic covering of the affected areas of skin.

4. A topical patch in accordance with claim 1, wherein the patch is designed to provide physical covering of the affected areas of skin.

5. A topical patch in accordance with claim 1, wherein the matrix has at least two layers.

6. A topical patch in accordance with claim 1, wherein the patch is furnished with a cover layer.

7. A topical patch in accordance with claim 1, wherein the patch is furnished with a protective layer.

8. A topical patch in accordance with claim 1, wherein the patch is designed as a lip patch.

9. A topical patch in accordance with claim 1, wherein at least part of the area of the patch is self-adhesive.

10. A patch in accordance with claim 1, wherein it contains at least aciclovir as an active substance.

11. A patch in accordance with claim 1, wherein it contains at least penciclovir as an active substance.

12. A patch in accordance with claim 1, wherein it contains one or more active ingredients from the group comprising docosanol, tromcardin HCl, zinc sulfate, heparin Na, silicic acid, melissa extract, and extract of Echinacea species.

13. A patch in accordance with claim 1, wherein it additionally contains one or more other active ingredients for other types of indications.

14. A patch in accordance with claim 1, wherein it additionally contains dexpanthenol.

15. A patch in accordance with characterized by claim 1, comprising a maximum patch surface area of <4 cm², preferably <2.5 cm², and especially <1.5 cm².

16. A patch in accordance with claim 1, wherein the patch can be cut by the patient to adjust its size according to individual needs.

17. A patch in accordance with claim 1, comprising a flexible, impermeable cover layer and a removable protective layer.

18. A patch in accordance with claim 1, comprising a cover layer based on at least one material from the group comprising polyester, ethylene-vinyl acetate, polyurethane, polypropylene, or polyethylene, each of which may possibly be pigmented.

19. A patch in accordance with claim 1, wherein the cover layer has a thickness of 10-100 μm, preferably 20-80 μm, and especially 25-60 μm.

20. A patch in accordance with claim 1, wherein makeup can be applied to the surface of the cover layer.

21. A patch in accordance with claim 1, comprising a removable protective layer based on at least one material from the group comprising polyester, polypropylene, polysiloxane, polyacrylate, ethylene-vinyl acetate, polyurethane, polyisobutene, or paper coated with silicone and/or polyethylene.

22. A patch in accordance with claim 1, comprising a matrix layer based on silicone.

23. A patch in accordance with claim 1, comprising a matrix layer based on polyacrylate.

24. A patch in accordance with claim 1, comprising a matrix layer based on at least one material from the group comprising homopolymers of acrylic acid crosslinked with allyl sucrose or allyl pentaerythritol, homopolymers of acrylic acid crosslinked with divinyl glycol, or copolymers of acrylic acid with small amounts of long-chain alkyl acrylate comonomers crosslinked with allyl pentaerythritol.

25. A patch in accordance with claim 1, comprising a matrix layer based on at least one material from the group comprising polyisobutylene, rubber, rubber-like synthetic homopolymers, copolymers or block polymers, butyl rubber, styrene-isoprene copolymer, polyurethanes, copolymers of ethylene, polysiloxanes, or styrene-butadiene copolymer.

26. A patch in accordance with claim 1, comprising a matrix layer based on a combination of at least two matrix formers.

27. A patch in accordance with claim 1, wherein the matrix has adhesive properties than guarantee that the patch can be worn on the skin, especially the lip, for at least 8 hours.

28. A patch in accordance with claim 1, wherein at the end of the period of application, it can be removed easily and painlessly from the skin, preferably from the lip, and especially preferably from the lesion.

29. A patch in accordance with claim 1, wherein a foreign body sensation is avoided as much as possible by a suitable choice of cover layer and matrix.

30. A patch in accordance with claim 1, comprising a permeation promoter, especially a substance selected from the following group and/or all mixtures thereof: monovalent and/or polyvalent aliphatic, alicyclic, and/or aromatic-aliphatic alcohols with up to eight C atoms each, e.g., ethanol, 1,2-propanediol, dexpanthenol, and/or polyethylene glycol; alcohol/water mixtures; saturated and/or unsaturated fatty alcohols with 8-18 C atoms each; terpenes, e.g., cineol, carveol, menthone, terpineol, verbenone, menthol, limonene, thymol, cymene, terpinene-4-ol, neomenthol, geraniol, and fenchone; mixtures of terpenes and ethanol and/or propylene glycol; tea tree oil; saturated and/or unsaturated cyclic ketones; alkyl methyl sulfoxides; saturated and/or unsaturated fatty acids with 8-18 C atoms each; their esters and salts; natural vitamin E; synthetic vitamin E and/or vitamin E derivatives; sorbitan fatty acid esters and ethoxylated sorbitan fatty acid esters; azones (laurocapram); azones mixed with alcohols; urea; 1-alkylpyrrolidone; block copolymers of polyethylene glycol and dimethylsiloxane with a cationic group at one end; folate polyethylene glycol liposome, proliposome; polyoxyethylene-10-stearyl ether; a mixture of polyoxyethylene-10-stearyl ether and glyceryl dilaurate; dodecyl-2-(N,N-dimethylamino)-propanol tetradecanoate and/or dodecyl-2-(N,N-dimethylamino)-propionate; N-acetylprolinate esters with more than 8 C atoms; nonionic surfactants, e.g., lauryl ether, esters of polyoxyethylene; ethosome (phospholipids vesicle); dimethyl(arylimino)sulfurane; a mixture of oleic acid analogs and propylene glycol; and a mixture of Padimate O, octyl salicylate, octyl methoxycinnamate, and laurocapram.

31. A patch in accordance with claim 1, wherein the carrier layer is semipermeable.

32. A patch in accordance with claim 1, wherein the matrix has a content of active ingredients of at least 40 wt. %.

33. A patch in accordance with claim 1, wherein, although the matrix has an active ingredient content of at least 40 wt. %, it also has a high level of adhesive strength.
